(19) United States
(12) Patent Application Publication
(10) Pub. No.: US 2014/0170128 A1
(43) Pub. Date: Jun. 19, 2014

(54) COMPOSITIONS FOR THE MANAGEMENT OF GLYCATED HEMOGLOBIN AND BLOOD GLUCOSE

(71) Applicant: VIJAI K. PASUPULETI, GENEVA, IL (US)

(72) Inventor: VIJAI K. PASUPULETI, GENEVA, IL (US)

(21) Appl. No.: 13/717,305

(22) Filed: Dec. 17, 2012

(51) Int. Cl. A61K 38/03; A61K 33/06; A61K 31/4188; A61K 31/385; A61K 36/54; A61K 33/24; A61K 38/16

(52) U.S. Cl. CPC A61K 38/03; A61K 33/24; A61K 33/06; A61K 38/16; A61K 31/385; A61K 36/54; A61K 31/4188
USPC 424/94.1; 424/655; 514/387

(57) ABSTRACT

Disclosed herein are compositions, products, and methods that treat glycated hemoglobin- and blood glucose-affected conditions by improving insulin function for the control and maintenance of blood glucose levels, alleviating oxidative stress, minimizing the risk of developing disease complications, facilitating the loss of weight and maintenance of healthy weight, and at the same time fulfill cravings for pleasant-tasting items of consumption and in turn increase the likelihood of patient compliance with the relevant therapy regimen. The disclosed compositions, products, and methods implement proteins and peptides that provide insulinotropic metabolic products in a sustained manner, in combination with ingredients that, for example, provide sustained release of carbohydrates, steady energy, sustained antioxidant effect relieve oxidative stress, and promote satiety, in order to reduce A1c and blood glucose levels and avert weight gain among subjects in need.

### TECHNICAL FIELD

The present disclosure pertains to the treatment of glycated hemoglobin- or blood glucose-affected conditions.

### BACKGROUND

There are several types of diabetes, although diabetes Types 1 and 2 are the most prevalent. Type 1 diabetes (T1D) can occur at any age, but most commonly is diagnosed from infancy to the late thirties. While type 2 diabetes (T2D) predominates among middle-aged individuals, recent studies have demonstrated that it also occurs in young adults who are overweight or obese. In Type 1 diabetes, insulin secretion is deficient. In Type 2 diabetes, insulin secretion may be sufficient, but the secreted insulin is resistant to glucose; to date researchers do not understand the precise mechanism underlying such resistance.

Diabetes may be characterized by various symptoms. The most common symptoms include thirst, frequent urination, perspiration, and fatigue, and the type and intensity of symptoms may vary from individual to individual. Indeed, early diagnosis of diabetes is often difficult because symptoms are not sufficiently prominent to hamper daily activities.

Type 2 diabetes progresses over an extended period of time and is typically preceded by a phase referred to as “prediabetes” that can last several years. In 2002, the Department of Health and Human Services (DHHS) and American Diabetes Association (ADA) introduced the term “prediabetes” as a substitute for the former clinical terms “impaired glucose tolerance” and “impaired fasting glucose”. The purpose of this change in terminology was to highlight the seriousness of the condition and to motivate afflicted individuals to pursue appropriate treatment. Individuals with prediabetes have blood glucose levels that are higher than in the healthy state, but not high enough to permit a diagnosis of actual diabetes. A fasting plasma glucose value between 100 to 125 mg/dl or more indicates impaired fasting glucose (IFG). In accordance with the oral glucose tolerance test, plasma glucose values between 140 to 199 mg/dl at 2 hours post-glucose load indicates impaired glucose tolerance (IGT).

According to the recently released CDC National Diabetes Fact Sheet 2011 (Centers for Disease Control and Prevention. National diabetes fact sheet: national estimates and general information on diabetes and prediabetes in the United States, 2011. Atlanta, Ga.: U.S. Department of Health and Human Services, Centers for Disease Control and Prevention, 2011), 25.8 million individuals in the United States are suffering from diabetes, and 79 million have prediabetes. The estimated total cost for the treatment of diabetes in 2010 was $174 billion. With a growing proportion of the population in the United States qualifying as overweight or obese, it can be expected that the number of individuals that will be afflicted with diabetes and prediabetes, as well as the treatment costs, will only increase. Thus, the need for treatment modalities that are effective to alleviate the physical and economic impacts of diabetes, prediabetes, and related disorders will become even more acute over time.

The Diabetes Fact Sheet and related literature also report that damage to the body commences during the prediabetes stage. In other words, blood sugar levels rise as the insulin resistance gradually develops, resulting in a familiar series of health problems, including high blood pressure, increased risk of heart attacks, neuropathy, nephropathy, and blindness.

A further parameter for diagnosing prediabetes and assessing identified cases of diabetes is A1C number (also referred to as “glycated hemoglobin”, “hemoglobin A1C”, or “HbA1c”), which correlates to the average amount of glucose in the blood during a certain period of time (for example, three to four months) preceding the test. The higher a patient's A1C level, the poorer the blood sugar control. An A1C level that falls within the range of 5.7% to 6.4% is indicative of prediabetes. If the patient had previously been diagnosed with diabetes, higher A1C levels correspond to a higher risk of developing diabetes complications. For most individuals with diabetes, the objective is to maintain an A1C result of less than 7. An A1C result that is greater than 7 corresponds to a higher risk of developing such diabetes complications as eye disease, kidney disease, heart disease, and nerve damage. A patient that succeeds in lowering the A1C test result can improve his or her chances of remaining healthy.

According to the Diabetes Control and Complications Trial (DCCT) conducted from 1983 to 1993 and the follow up study, Epidemiology of Diabetes Interventions and Complications (EDIC), for every point reduction in A1C level, eye disease risk is reduced by 76%, kidney diseases risk is reduced by 50%, nerve disease risk is reduced by 60%, cardiovascular disease event risk is reduced by 42%, and non-fatal heart attack, stroke, or risk of death from cardiovascular causes is reduced by 57%. However, in 2008 it was reported that about 40% of patients nationwide (approximately 9 million people) were not in conformity with an A1C goal of <7 while taking oral antidiabetic drugs alone (see Hoerger T J, et al., Diabetes Care. 2008; 31(1):81-86). Extrapolating these results to the 2012 population size means that the number of diabetic individuals who do not elicit an adequate response to oral antidiabetic drugs approaches 11 million. Therapeutic methodologies that permit patients to lower their A1C test results would therefore improve the chances of avoiding health complications of diabetes for millions of patients in the United States.

Recent findings clearly demonstrate that the etiology of diabetes is multifactorial, with each factor acting independently and possibly synergistically to induce the traditional symptoms of the disease. Accordingly, it is unsurprising that there is no single pharmaceutical treatment option, other than insulin itself, for alleviation of such symptoms. To date there is no FDA-approved drug for preventing insulin resistance and prediabetes. Diabetes is a progressive disease, and to date most patients have required a combination of two or three anti-hyperglycemic agents in order to attain satisfactory glucose control in the long-term. Typically, when the traditional first two choices—metformin and sulfonyl urea—fail, the third choice is selected from alpha-glucosidase inhibitors, thiazolidinediones, glucagon-like peptide-1 (GLP-1) agonists, dipeptidyl peptidase-4 (DPP-4) inhibitors, and insulin. Only a few studies have considered the effect of adding a third antihyperglycemic drug when blood glucose control is not achieved by using metformin and a sulfonylurea. Recently Gross et al. (Ann Intern Med. 2011; 154:672-67), conducted a meta-analysis study that compared the efficacy of add-on antihyperglycemic drugs in patients with type 2 diabetes that is not controlled with metformin and a sulfonylurea. The study concluded that there is no clear difference in benefit between drug classes when adding a third agent to treatment of patients with type 2 diabetes who are already receiving metformin and a sulfonylurea. In addition, the Gross, et al. study observed that a problematic side effect of administering third line drugs is weight gain, which is particularly common among individuals taking insulin or a thiazolidinedione. According to the study, the average weight gain for those on insulin was about six pounds, and the average weight gain for patients taking thiazolidinedione was more than nine pounds. Weight gain therefore remains a vexing side effect of numerous prominent pharmaceutical treatments for diabetes.

Several approaches are available as alternatives or adjuncts to pharmaceutical intervention. One important outcome of the research associated with the landmark Diabetes Prevention Program, a major multicenter clinical research study, is that diabetes can be prevented among high-risk adults by intensive lifestyle modification, including healthy diet and exercise (see Knowler W C, et al., N Engl J Med. 2002 Feb. 7; 346(6):393-403). Among such individuals, over a three year study period 58% successfully prevented disease onset by implementing lifestyle changes, as compared with a success rate of 31% among individuals who used the medication metformin (Id.).

However, such approaches are not successfully translated in clinical practice for a significant proportion of high-risk individuals and individuals with prediabetes or diabetes. An urgent need remains for additional strategies for reducing the risk of entering the prediabetic or diabetic state, and for mitigating or abating the disease symptom profile or potential of those already on the diabetes spectrum.

### SUMMARY

Disclosed are compositions for modulating the level of glycated hemoglobin or blood glucose in a subject. The compositions may include at least two proteins that undergo human metabolism at different rates, each forming insulinotropic metabolic products (including amino acids, di-peptides, and tri-peptides) upon human metabolism; at least two peptides that undergo human metabolism at different rates, each forming insulinotropic metabolic products upon human metabolism; at least one insulinotropic amino acid; a biologically acceptable chromium-containing material; a biologically acceptable magnesium-containing material; and at least one antioxidant.

Also provided are powdered beverage mixtures, liquid beverages, and solid food items comprising a composition for modulating the level of glycated hemoglobin or blood glucose in a subject.

The present application also discloses methods for treating a glycated hemoglobin- or blood glucose-affected condition in a subject comprising administering to the subject a composition comprising at least two proteins that undergo human metabolism at different rates, each forming insulinotropic metabolic products (including amino acids, di-peptides, and tri-peptides) upon human metabolism; at least two peptides that undergo human metabolism at different rates, each forming insulinotropic metabolic products upon human metabolism; at least one insulinotropic amino acid; a biologically acceptable chromium-containing material; a biologically acceptable magnesium-containing material; alpha lipoic acid; at least one antioxidant; and, dietary fiber.

### BRIEF DESCRIPTION OF THE DRAWINGS

FIG. 1 provides the results of a comparison between the effect of an exemplary embodiment of the inventive compositions and the effect of a placebo composition on A1C test values in (A) all test subjects, (B) test subjects stratified to >8.5 A1C, and (C) test subject stratified to <8.5 A1C.

FIG. 2 provides the results of a comparison between the effect of an exemplary embodiment of the inventive compositions and the effect of a placebo composition on fasting blood glucose levels in (A) all test subjects, (B) test subjects stratified to >8.5 A1C, and (C) test subject stratified to <8.5 A1C.

FIG. 3 provides the results of a comparison between the effect of an exemplary embodiment of the inventive compositions and the effect of a placebo composition on patient weight in (A) all test subjects, (B) test subjects stratified to >8.5 A1C, and (C) test subject stratified to <8.5 A1C.

### DETAILED DESCRIPTION OF ILLUSTRATIVE EMBODIMENTS

The presently disclosed inventions may be understood more readily by reference to the following detailed description taken in connection with the accompanying figures and examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

In the present disclosure the singular forms “a,” “an,” and “the” include the plural reference, and reference to a particular numerical value includes at least that particular value, unless the context clearly indicates otherwise. Thus, for example, a reference to “an antioxidant” may be a reference to one or more of such substances and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as including the option of specifying “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

Unless otherwise specified, any component, element, attribute, or step that is disclosed with respect to one embodiment of the present methods and products may apply to any other method or product that is disclosed herein.

The disclosures of each patent, patent application, and publication cited or described in this document are hereby incorporated herein by reference, in their entirety.

There is basic consensus that lifestyle changes and oral medication, such as metformin, should represent first-line treatment for patients with type 2 diabetes. However, 55 to 70% of patients who initially achieve their glycemic targets with metformin therapy experience a progressive deterioration of glucose control during the two to three years that follow. Sulfonylureas are a commonly used second medication on the basis of efficacy, availability, and cost. However, adding a sulfonylurea to metformin therapy (or vice versa) usually does not maintain long-term control, and deterioration develops in as soon as six months. HbA1c levels typically also rise among patients who experience deterioration of glucose control following positive lifestyle changes, one or more oral medication regimens, or both. Furthermore, although adjunct oral medication therapy for diabetes may permit temporary glucose control, weight gain is a common side effect among patients that are administered second- and third-line medications, especially in the case of insulin therapy.

Reduction in HbA1c levels is especially critical among those with prediabetes or diabetes for the maintenance of good health, which in turn influences the ability of a subject to maintain a normal lifestyle and avoid complications that often cascade into a debilitating and costly physical condition. For example, a patient that is able to lower his or her measured HbA1c value by 1 (e.g., from 9 to 8) may be able to reduce the likelihood of developing diabetes-induced health problems by as much as 40%. A therapeutic composition that can assist a subject to lower his or her HbA1c level, even by a small margin, would be of considerable value from an individual and societal perspective.

Patient compliance is also an important component of any therapy regimen. This is especially the case among subjects who are diabetic or are in peril of developing the disease, who in certain instances may have engaged in risky dietary practices, and therefore may be averse to adopting lifestyle changes that present physical challenges. A therapeutic composition that can be presented in a form that is pleasant to consume can increase the likelihood that a patient will maintain a prescribed program for minimizing diabetic risk.

Disclosed herein are compositions, products, and methods that treat insulin resistance, prediabetes, type 2 diabetes and metabolic syndrome symptoms, by improving insulin function for the control and maintenance of blood glucose levels, minimizing risk of developing diabetes complications, facilitating the loss of weight and maintenance of healthy weight, and at the same time fulfill cravings for pleasant-tasting items of consumption and in turn increase the likelihood of patient compliance with the relevant therapy regimen.

Significantly, the present compositions, products that contain them, and methods involving their administration provide therapeutically meaningful improvements in numerous respects, including reduction in A1C values, reduction in fasting blood glucose levels, facilitation of weight loss, lowered cholesterol levels, increased levels of incretins, and significant decrease in systolic and diastolic blood pressure. The inventive compositions may also be administered in a number of ways that encourage patient compliance with an intake regimen, such as in nutritional foods and drinks. In addition, the present compositions, products, and methods may be utilized in combination with pharmaceutical intervention and healthy lifestyle changes in order to provide the therapeutic improvements listed above (among others) that is additive and in some instances synergistic with the therapies to which they are adjunct. As described more fully herein, upon contact with a subject's digestive system, the compositions of the present disclosure permit sustained absorption of insulinotropic metabolic products (including amino acids, di-peptides, and tri-peptides) and carbohydrates, which thereby activates incretins to regulate the blood glucose levels during and after the period during which the compositions are digested. Thus, the present compositions may be said to represent a sustained source of insulinotropic metabolic products from proteins and peptides, a sustained source of glucose from complex carbohydrates, and a sustained source of antioxidants that regenerate their oxidized forms and provide sustained antioxidant potential to relieve oxidative stress.

Disclosed are compositions for modulating the level of glycated hemoglobin or blood glucose in a subject. The compositions may include at least two proteins that undergo human metabolism at different rates, each forming one or more insulinotropic metabolic products upon human metabolism; at least two peptides that undergo human metabolism at different rates, each forming insulinotropic metabolic products (including amino acids, di-peptides, and tri-peptides) upon human metabolism; at least one insulinotropic amino acid, oligopeptide, tri-peptide, or di-peptide; a biologically acceptable chromium-containing material; a biologically acceptable magnesium-containing material; and, at least one antioxidant. In certain embodiments, the compositions may additionally comprise alpha lipoic acid, dietary fiber, or both.

As used herein, an “insulinotropic metabolic product” of a protein or of a peptide is any insulinotropic substance that results from the partial or complete human metabolism of the protein or peptide. Exemplary insulinotropic metabolic products of proteins and peptides include oligopeptides (peptides that include more than three amino acids), tri-peptides (peptides that include three amino acids), and di-peptides (peptides that include two amino acids). An “insulinotropic protein” is a protein that is itself insulinotropic or that results in one or more insulinotropic metabolic products upon undergoing human metabolism. An “insulinotropic peptide” is a peptide that is itself insulinotropic or that results in one or more insulinotropic metabolic products upon undergoing human metabolism.

The present compositions may include any two proteins that respectively undergo human metabolism at different rates and that are metabolized into one or more insulinotropic metabolic products (including amino acids, di-peptides, tri-peptides, and oligopeptides). It has been discovered that the inclusion of such proteins provides temporally offset release of insulinotropic metabolic products, and that this release profile permits a sustained insulinotropic effect. The rate of metabolic conversion of proteins to insulinotropic metabolic products, such as the conversion of proteins to peptides, and then from peptides to amino acids, naturally varies from protein to protein. The present compositions utilize this natural effect in a novel way in order to provide what is effectively controlled release of one or more insulinotropic metabolic products and thereby an insulinotropic effect that is sustained and long-lasting rather than a burst-type profile.

Exemplary proteins for inclusion in the present compositions include vegetable, legume, tuber, or grain proteins (e.g., soy, pea, rice, or potato), whey protein, casein, and salts of casein. Whey protein and whey protein isolate are examples of insulinotropic proteins that may be used in the present compositions. Whey protein isolate may have a protein range of, for example, from about 80% to about 95%. Soy protein and soy protein isolate are further examples of insulinotropic proteins that may be used in the present compositions. Soy protein isolate may have a protein range of, for example, from about 80% to about 92%. The present compositions may provide soy protein in the form of, for example, soy milk powder, soy protein isolate, or both. Soy milk powder may be substituted with whole fat, reduced fat, or defatted soy milk. In some embodiments, soy milk powder may be replaced with soy protein isolate. Casein and salts thereof represent additional examples of insulinotropic proteins that may be used in the present compositions. In the case of caseinate, the protein range may be, for example, from about 75% to about 90%. Sodium caseinate, potassium caseinate, and calcium caseinate each represent alternative forms of caseinate that may be used in the present compositions. Animal and vegetable proteins are additional classes of insulinotropic proteins that may be used. Exemplary animal proteins or sources of animal proteins include collagen, gelatin, meat, fish, and egg. Vegetable proteins include, for example, gluten, pea, potato, rice, hemp, and yeast proteins. In addition to providing controlled release of insulinotropic metabolic products, the proteins increase satiety, which assists in controlling hunger and prevents overeating that can lead to weight gain, the implications of which for diabetic patients, for example, are well known. Furthermore, the proteins may provide increased levels of incretins (of which GLP-1 and GIP are examples), which regulate glucose metabolism by an increase in the amount of insulin released from the beta cells of the islets of Langerhans after eating.

In the present compositions, there may be two or more of the proteins that respectively undergo human metabolism at different rates and that are metabolized into insulinotropic metabolic products (including amino acids, di-peptides, and tri-peptides). For example, the compositions may include two, three, four, five, six, seven, eight, nine, ten or more separate proteins. When more than two proteins are present, at least two of the proteins respectively undergo human metabolism at different rates, and two or more of the proteins that are present may undergo human metabolism at substantially similar rates. Alternatively, when there are more than two proteins in a given composition, each of the proteins may respectively undergo human metabolism at different rates.

As used herein, a protein or peptide that has “undergone human metabolism” has been at least partially broken down into its constituent insulinotropic metabolic products (including amino acids, di-peptides, and tri-peptides) through the process of human metabolism. Preferably, at least 75%, 80%, at least 85%, at least 90%, at least 95%, or all of the protein or peptide has been broken down into its constituent insulinotropic metabolic products pursuant to the process of undergoing human metabolism. When two proteins (or two peptides) respectively undergo human metabolism at different rates, this means that it takes a longer period of time for one protein (or peptide) to break down into its constituent insulinotropic metabolic products to a certain degree following ingestion by the subject than it takes the other protein (or peptide) to break down to the same degree. For example, if 95% of one protein (or peptide) breaks down into its constituent insulinotropic metabolic products 30 minutes after ingestion, then a second protein (or peptide) undergoes human metabolism at a slower rate if it takes more than 30 minutes for 95% of the second protein (peptide) to break down into its constituent insulinotropic metabolic products. A given protein (or peptide) may undergo human metabolism in less than 6 hours, less than 5 hours, less than 4 hours, less than 3 hours, less than 2 hours, less than 1 hour, less than 45 minutes, less than 30 minutes, less than 20 minutes, less than 15 minutes, or less than 10 minutes.

In certain embodiments, the composition according to the present disclosure may comprise a first protein that undergoes human metabolism 30 minutes or less following administration to the subject, and a second protein that undergoes human metabolism more than 30 minutes following administration to a subject, but less than, for example, 4 hours, 3 hours, 2 hours, or 1 hour. In some embodiments, the composition comprises three proteins that undergo human metabolism at respectively different rates, such a first protein that undergoes human metabolism less than 30 minutes following administration to a subject, a second protein that undergoes human metabolism more than 30 minutes following administration but less than 2 hours following administration, and a third protein that undergoes human administration more than 2 hours following administration.

In some embodiments, the proteins include at least one of soy protein, whey protein, and casein or a salt thereof. For example, the composition may comprise soy protein, whey protein, and casein or casienate salts.

The total amount of the proteins in the compositions that are disclosed herein may be from about 25% by weight to about 80% by weight, preferably from about 30% by weight to about 70% by weight, to about 35% by weight to about 65% by weight, or about 40% by weight to about 60% by weight of the composition.

The present compositions may include any two peptides that respectively undergo human metabolism at different rates and that are metabolized into insulinotropic metabolic product. As in the case of the above-described proteins, the release profile of the peptides permits a sustained insulinotropic effect. The rate of metabolic conversion of peptides to insulinotropic metabolic products naturally varies from peptide to peptide. For example, casein hydrolysate digests with higher degree of hydrolysis and absorbs faster than whey and soy hydrolysates with lower degree of hydrolysis. The rate of conversion of peptides to amino acids may depend on the absorption rate of a given amino acid, which in turn depends on the degree of hydrolysis.

The present compositions utilize this natural effect in a novel way in order to provide what is effectively controlled release of insulinotropic metabolic products and thereby an insulinotropic effect that is sustained and long-lasting rather than a burst-type profile. Exemplary peptides for inclusion in the present compositions include hydrolysates of any previously-listed protein (such as peptides from vegetable, legume, tuber, or grain proteins—e.g., soy, pea, rice, or potato—whey protein, casein or salts thereof, such as caseinate) and glutathione. Whey protein hydrolysate is also referred to herein as bioactive peptides from whey, and may contain a protein range of, for example, about 40% to about 85%, and may have a degree of hydrolysis of, for example, about 8% to about 24%. Soy protein hydrolysate is also referred to herein as bioactive peptides from soy, and may contain a protein range of, for example, about 40% to about 85%, and may have a degree of hydrolysis of, for example, about 8% to about 24%. Casein hydrolysate is also referred to herein as bioactive peptides from casein, and may contain a protein range of, for example, about 75% to about 85%, and may have a degree of hydrolysis of, for example, about 8% to about 45%. Like the proteins of the present composition, the peptides can increase satiety.

In the present compositions, there may be two or more of the peptides that respectively undergo human metabolism at different rates and that are metabolized into insulinotropic metabolic products. For example, the compositions may include two, three, four, five, six, seven, eight, nine, ten or more separate peptides. When more than two peptides are present, at least two of the peptides respectively undergo human metabolism at different rates, and two or more of the peptides that are present may undergo human metabolism at substantially similar rates. Alternatively, when there are more than two peptides in a given composition, each of the peptides may respectively undergo human metabolism at different rates.

In certain embodiments, the composition according to the present disclosure may comprise a first peptide that undergoes human metabolism 30 minutes or less following administration to the subject, and a second peptide that undergoes human metabolism more than 30 minutes following administration to a subject, but less than, for example, 4 hours, 3 hours, 2 hours, or 1 hour. In some embodiments, the composition comprises three peptides that undergo human metabolism at respectively different rates, such a first peptide that undergoes human metabolism less than 30 minutes following administration to a subject, a second peptide that undergoes human metabolism more than 30 minutes following administration but less than 2 hours following administration, and a third peptide that undergoes human administration more than 2 hours following administration.

In some embodiments, the at least two peptides are respectively metabolic products of the at least two proteins. For example, the composition may comprise two or more of soy protein hydrolysate, casein hydrolysate, and whey protein hydrolysate. In another embodiment, the composition may comprise any two of soy protein hydrolysate, casein hydrolysate, whey protein hydrolysate, and glutathione.

The total amount of the peptides in the compositions that are disclosed herein may be from about 5% by weight to about 40% by weight, from about 5% by weight to about 30% by weight, to about 10% by weight to about 30% by weight, or about 15% by weight to about 30% by weight of the composition.

The present compositions also include at least one insulinotropic amino acid. The inclusion of at least one such amino acid provides “instant release” of an insulinotropic ingredient, such that the insulinotropic effect of the present compositions occurs as soon as metabolism commences. In combination with the above-described peptides and proteins, the amino acids provide prolonged insulinotropic effect that begins as soon as the subject begins digesting the contents of the composition. Insulinotropic amino acids include, for example, phenyalanine, leucine, isoleucine, valine, lysine, threonine, arginine, and 4-hydroxyisoleucine. The total amount of the insulinotropic amino acids in the compositions that are disclosed herein may be from about 0.1% by weight to about 5% by weight, from about 0.2% by weight to about 4.5% by weight, to about 0.25% by weight to about 4% by weight, or about 0.3% by weight to about 4% by weight of the composition.

A biologically acceptable chromium-containing material is an additional ingredient of the present compositions. Micronutrients play direct and indirect roles in assisting to alleviate prediabetic and diabetic symptoms and increasing the insulin function. Several studies have reported that chromium supplements can lead to normal or more rapid clearance of glucose from the blood. Any biologically acceptable source of chromium (i.e., that, upon ingestion, safely delivers chromium to the body) may be used in accordance with the present compositions. For example, brewer's yeast grown in the presence of chromium is bioavailable and offers a similar ability to regulate blood sugar levels. In addition the vitamins in yeast will act in synergy with chromium to reduce and control glucose levels. Other sources of chromium include, for example, chromium chloride and chromium picolinate. The total amount of chromium in the present compositions may be from about 0.2% by weight to about 5% by weight, from about 0.3% by weight to about 4% by weight, to about 0.4% by weight to about 3.5% by weight, or about 0.35% by weight to about 3% by weight of the composition.

The present compositions also include a biologically acceptable magnesium-containing material. Magnesium is apparently necessary for the secretion and release of insulin, and is also required by cells for maintaining insulin sensitivity and increasing the number of insulin receptors. Without adequate magnesium levels within body cells, insulin becomes less effective. It is believed that a deficiency of magnesium interrupts insulin secretion in the pancreas and increases insulin resistance in tissue. Evidence suggests that a deficiency of magnesium may contribute to certain diabetes complications. Any biologically acceptable source of magnesium may be used in accordance with the present compositions. For example, dimagnesium malate, magnesium oxide, magnesium citrate, and amino acid chelates of magnesium each represent acceptable forms of that mineral. The total amount of magnesium in the present compositions may be from about 1% by weight to about 15% by weight, from about 1% by weight to about 10% by weight, to about 1% by weight to about 7% by weight, or about 1% by weight to about 5% by weight of the composition.

A further ingredient of the present compositions may be alpha lipoic acid. Alpha lipoic acid is an antioxidant and is important in the regulation of chronic disease because of its ability to destroy free radicals and regenerate other antioxidants to help lower blood sugar levels. Alpha lipoic acid exists in two forms, one is a mixture of the R and S isomers, and the other is pure R isomer. The present compositions may include either or both forms. The total amount of alpha lipoic acid in the present compositions may be from about 0.3% by weight to about 5% by weight, from about 0.35% by weight to about 4.5% by weight, to about 0.4% by weight to about 4% by weight, or about 0.5% by weight to about 3.5% by weight of the composition.

The present compositions include at least one natural or synthetic antioxidant in addition to alpha lipoic acid (which is itself an antioxidant). Preferably, the antioxidant is one that is capable of regenerating its oxidized forms and providing sustained antioxidant potential to relieve oxidative stress. The additional antioxidant may be, for example, one or more antioxidant vitamins, such as vitamin C or vitamin E. Other examples include peptides (such as glutathione) and natural extracts that possess antioxidant properties. Some embodiments of the present compositions include at least one fat-soluble antioxidant vitamin and at least one water-soluble antioxidant vitamin. Vitamins provide energy. At the same time, it has been demonstrated that that water soluble vitamins are depleted in diabetic patients because of frequent urination. Furthermore, additional studies have shown that long term use of metformin (which is widely prescribed as first-line drug) depletes vitamin B12. Vitamin E may assist in the prevention of heart and kidney problems by improving circulation, decreasing the oxidized form of LDL cholesterol, and decreasing the likelihood of clots, which are common in diabetics. Vitamin E also improves glucose tolerance in most type 2 diabetics, and it has been discovered that oral vitamin E treatment can contribute to the normalization of retinal (blood flow) abnormalities and improving kidney function. The total amount of additional antioxidant in the present compositions may be about 0.2% by weight to about 7% by weight, about 0.25% by weight to about 6% by weight, about 0.3% by weight to about 4% by weight, or about 0.3% by weight to about 3.5% by weight. For example, vitamin E may be present in the amount of about 0.25% by weight to about 2.5% by weight, and vitamin C may be present in the amount of about 0.30% by weight to about 3% by weight.

Additional vitamins may also be included in the present compositions. For example, any water-soluble or fat soluble vitamin may be included in order to assist in alleviating oxidative stress, provide energy, and restore vitamins that are depleted due to diabetic symptoms such as frequent urination. Exemplary vitamins include vitamin B6, vitamin B12, and biotin. The total amount of additional vitamins in the present compositions may be about 0.0000001% by weight to about 0.4% by weight, about 0.000001% by weight to about 0.3% by weight, or from about 0.00001% by weight to about 0.25% by weight. For example, vitamin B6 may be present in the amount of about 0.02% by weight to about 0.20% by weight, vitamin B12 may be present in the amount of about 1.0E-8% by weight to about 1.0E-5% by weight, and biotin may be present in the amount of about 0.006% by weight to about 0.06% by weight.

In some embodiments, the present compositions include vitamin D, vitamin E, or both. In other embodiments, the compositions include one or more of vitamin B6, vitamin B12, vitamin C, and biotin. Certain compositions include all of vitamin D, vitamin E, vitamin B6, vitamin B12, vitamin C, and biotin.

The present compositions may also include dietary fiber. For example, the compositions may include a source of soluble fiber, a source of insoluble fiber, or both. Examples of dietary fiber include resistant maltodextrin and resistant starch. Other examples include fructooligosaccharides, galactooligosaccharides, and mannanoligosaccharides. The total amount of dietary fiber in the present compositions may be from about 2% to about 25% by weight, about 4% to about 20% by weight, about 5% to about 15% by weight, or about 7% to about 12% by weight. The complex carbohydrates in dietary fiber can slow down the absorption of sugar and provide energy in a controlled fashion. In addition, they can assist with the maintenance of healthy intestinal flora, which is in turn beneficial to the immune system.

The compositions according to the present disclosure may also include a source of a polyphenol. An exemplary source of polyphenols is cinnamon or cinnamon extract. For example, the present compositions may include whole cinnamon powder, cinnamon powder 10:1 extracts, cinnamon powder 1:4 extracts, water soluble cinnamon extract, Cinnamon may significantly help subjects with non-insulin dependent, type 2 diabetes improve their ability to respond to insulin, thus normalizing their blood sugar levels. The total amount of a source of a polyphenol may be about 0.05% by weight to about 5% by weight, about 0.08% to about 4% by weight, or about 1% by weight to about 3% by weight. In some embodiments, two or more sources of a polyphenol are present. For example, an exemplary composition may include both cinnamon powder and cinnamon extract, and in such embodiments, the cinnamon powder may be present in an amount of about 0.25% to about 2.5% by weight, and the cinnamon extract may be present in the amount of about 0.08% to about 0.75% by weight.

An exemplary embodiment in accordance with the present disclosure comprises as follows:

about 35 to about 65 wt % soy milk powder;

about 0.35 to about 4.5 wt % alpha lipoic acid;

about 0.20 to about 5 wt % of a biologically acceptable chromium-containing material;

about 1 to about 15 wt % of a biologically acceptable magnesium-containing material;

about 0.40 to about 3.5 wt % of at least one water-soluble antioxidant vitamin;

about 0.20 to about 2.5 wt % of at least one water-insoluble antioxidant vitamin;

about 2 to about 18.5 wt % whey protein hydrolysate;

about 2 to about 18.5 wt % whey protein isolate;

about 1 to about 10 wt % soy protein hydrolysate;

about 1 to about 10 wt % soy protein isolate;

about 1 to about 10 wt % casein hydrolysate;

about 1 to about 11 wt % casein or a salt thereof;

about 2 to about 4 wt % of at least one insulinotropic amino acid;

about 0.30 to about 3.5 wt % cinnamon powder or cinnamon extract; and,

about 2 to about 25 wt % dietary fiber.

An additional embodiment in accordance with the present disclosure comprises the following ingredients:

about 40 to about 60 wt % soy milk powder;

about 0.30 to about 3.5 wt % alpha lipoic acid R+S;

about 0.07 to about 0.60 wt % alpha lipoic acid R−NA;

about 0.40 to about 3.5 wt % of a biologically acceptable chromium-containing material;

about 1.2 to about 14 wt % of a biologically acceptable magnesium-containing material;

about 0.02 to about 0.20 wt % vitamin B6;

about 1.0E-8 to about 1.0E-5 vitamin B12;

about 0.25 to about 2.5 wt % vitamin E;

about 0.30 to about 3 wt % vitamin C;

about 0.006 to about 0.06 wt % biotin;

about 2 to 18 wt % whey protein hydrolysate;

about 2 to about 18.5 wt % whey protein isolate;

about 1 to about 9 wt % soy protein hydrolysate;

about 1 to about 9 wt % soy protein isolate;

about 1 to about 9 wt % casein hydrolysate;

about 1 to about 11 wt % casein or a salt thereof;

about 0.25 to about 2.5 wt % cinnamon powder;

about 0.08 to about 0.75 wt % cinnamon extract;

about 2 to about 25 wt % of a source of soluble fiber;

about 0.15 to about 1.5 wt % isoleucine; and,

about 0.15 to about 1.5 wt % phenylalanine.

In accordance with any of the preceding embodiments, the present compositions may include one or more of viscosity modifiers, sweeteners, flavoring agents, buffering agents, flow agents, and stabilizers.

Exemplary viscosity modifiers include microcrystalline cellulose, gums (e.g., cellulose gum), carrageenan, xanthan, pectin, and carboxymethylcellulose. Viscosity modifiers may be provided in the instant compositions in an amount of about 0.1% to about 2% by weight, preferably in an amount of about 0.15% by weight to about 1.6% by weight.

One or more sweeteners may be included in the present composition, for example, in order to improve palatability and satisfy cravings for sweet foods. Such sweeteners may be naturally-occurring or artificial. Exemplary sweeteners include aspartame, saccharine, xylitol, lactitol, mannitol, maltitol, sorbitol, isomalt, sucralose, erythritrol, acesulfame potassium, stevia, and stevia extracts. Sweeteners may be included in the present composition in an amount of about 0.50% to about 5.5% by weight.

Flavoring agents may also be included in the present composition in order to enhance palatability and satisfy cravings, and may be naturally-derived or artificial. Exemplary natural or artificial flavoring agents may impart a flavor that is vanilla, cinnamon, strawberry, blueberry, cherry, chocolate, orange, lemon, raspberry, or any other flavor that would be pleasant to a subject. Flavoring agents may be included in the present composition in an amount of about 0.2% to about 3.0% by weight.

The present compositions may also include one or more pharmaceutically acceptable buffering agents. The buffering agent may be suitable for the regulation of acidity or alkalinity. Exemplary buffering agents include potassium citrate, sodium citrate, potassium lactate, and tripotassium phosphate. Other suitable buffering agents will be readily identifiable by those of ordinary skill in the art. A buffering agent may be present in the present compositions in an amount of about 0.2% to about 3.0% by weight. For example, potassium citrate may be included in the present compositions in an amount of about 0.6% to about 1% by weight.

Flow agents may be included in the present composition, especially in embodiments wherein the composition is in the form of a powder, such as for use as a powdered beverage mixture. For example, silicon dioxide is a commonly-used flow agent in powdered food products. Flow agents may be provided in the instant compositions in an amount of about 0.1% to about 2% by weight, preferably in an amount of about 0.15% by weight to about 1.6% by weight.

The present compositions may also comprise one or more stabilizers. TIC Pretested® Colloid Ultrasmooth Powder (TIC Gums, White Marsh, Md.) represents an exemplary stabilizer. Stabilizers may be included in the present compositions in an amount of about 0.2% to about 2% by weight, or in an amount of about 0.5% to about 0.75% by weight.

The compositions may be packaged in any type of container or package known in the art for use in connection with food products, such as packaging formed in whole or in part from paper, glass, lined paperboard, plastic, or metal.

The presently disclosed compositions may be incorporated into any liquid, powdered, or solid food item, or a tablet, a capsule, or any other pharmaceutical dosage form, so that subjects may consume the composition in any desired form. For example, provided are powdered beverage mixtures, liquid beverages, or solid food items comprising a composition according the present disclosure. The solid food item may be, for example, an energy bar-type food item, a cookie, a pastry, a breakfast cereal, tortilla, flat bread, a yogurt, or a frozen dessert. Liquid beverages may, for example, take the form of an energy drink or shake. The selection of additional ingredients (i.e., those that are adapted for the particular food item or pharmaceutical dosage form and that are suitable for administration to a subject in whom it is desirable to modulate the level of glycated hemoglobin or blood glucose) and preparation of a liquid, powdered, or solid food item, or a tablet, a capsule, or any other pharmaceutical dosage form that includes a composition according to the present disclosure is within the ability and knowledge of those of ordinary skill in the art. In other embodiments, the present compositions may be prepared in liquid form for enteral or tube feeding.

The present disclosure also provides methods for treating a glycated hemoglobin- or blood glucose-affected condition in a subject, such methods comprising administering to said subject any composition according to the description provided above. In other words, the present methods comprise administering to the subject a composition comprising at least two proteins that undergo human metabolism at different rates, each forming one or more insulinotropic metabolic products upon human metabolism; at least two peptides that undergo human metabolism at different rates, each forming one or more insulinotropic metabolic products upon human metabolism; at least one insulinotropic amino acid, di-peptide, tri-peptide, or oligopeptide; a biologically acceptable chromium-containing material; a biologically acceptable magnesium-containing material; and at least one antioxidant vitamin, wherein the characteristics of such components may be according to any of the embodiments of the disclosed compositions that are described supra. The compositions that are administered to the subject pursuant to the present methods may further comprise any of the optional ingredients that are described above concerning the inventive compositions (such as, inter alia, dietary fiber and alpha lipoic acid). Furthermore, administration of “a composition” may include administration of an embodiment of the composition that is incorporated into any liquid, powdered, or solid food item, or a tablet, a capsule, or any other pharmaceutical dosage form. The incorporation of a composition according to the present disclosure into a liquid, powdered, or solid food item, or a tablet, a capsule, or any other pharmaceutical dosage form is described above and is applicable to the presently disclosed methods.

Because the actual act of causing the ingestion of the composition will in most cases be performed by the subject himself or herself, “administering” the composition to the subject is intended to embrace providing instructions to the subject to ingest the composition (such as might be done by a physician, physician's assistant, nurse, or any other health professional or consultant).

The administration of the composition according to the present disclosure may according to a specific schedule or treatment regimen. In certain embodiments, administration may occur several times per week, on a once-daily basis, or two or more times per day. The administration schedule may occur over a course of days, weeks, months, or years. For example, the present methods may comprise administering the composition (in any appropriate form) to the subject twice a day for one month, two months, three months, six months, nine months, one year, or indefinitely. During the administration period, the subject may undergo self-assessment or assessment by a medical professional to determine therapeutic progress. The administration regimen may be adjusted, for example, by increasing or decreasing number of daily or weekly administration episodes, depending on the results of the assessment.

The glycated hemoglobin- or blood glucose-affected condition in the subject to whom the composition is administered may be, for example, type 1 diabetes, type 2 diabetes, prediabetes, insulin resistance syndrome, metabolic syndrome, cystic fibrosis related diabetes, or polycystic ovarian syndrome. The subject may have been diagnosed with the glycated hemoglobin- or blood glucose-affected condition, may merely be suspected of having the condition, or may have been assessed to be at risk for the condition.

The administration of the composition may be as an adjunct therapy in combination with at least one other treatment for the glycated hemoglobin- or blood glucose-affected condition. As described more fully in the Examples that follow, it has presently been discovered that when the present compositions are administered to subjects who are already taking first- and second-line drugs for the management of diabetes (for example, metformin and glibenclamide) such subjects experience therapeutically meaningful improvements in numerous respects, including reduction in A1C values, reduction in fasting blood glucose levels, facilitation of weight loss, lowered cholesterol levels, increased levels of incretins, and significant decrease in systolic and diastolic blood pressure. This is to be contrasted with previous findings that although adjunct oral medication therapy for diabetes may permit temporary glucose control, progressive deterioration of glucose control, increase in HbA1c number, and weight gain are common long-term effects among patients that are administered second- and third-line medications. The administration of the present compositions may be adjunct to oral medicine therapy, insulin therapy, or any other first- and/or second-line therapy that is intended to treat the glycated hemoglobin- or blood glucose-affected condition.

In another embodiment, the present methods comprise administering a composition according to the present disclosure to a subject that is suffering from burns. It has been surprisingly discovered that the present compositions are effective to accelerate healing of burns when ingested by the subject who is suffering from such burns. The burns may be of any type (i.e., first-, second-, or third-degree) and origin (e.g., heat, chemical, sunlight). The administration of the composition according to such methods may be according to a specific schedule or treatment regimen. In certain embodiments, administration may occur several times per week, on a once-daily basis, or two or more times per day. The administration schedule may occur over a course of days, weeks, months, or years. For example, the present methods may comprise administering the composition (in any appropriate form) to the subject twice a day for one month, two months, three months, six months, nine months, one year, or as long as required pursuant to the healing process. During the administration period, the subject may undergo self-assessment or assessment by a medical professional to determine therapeutic progress. The administration regimen may be adjusted, for example, by increasing or decreasing number of daily or weekly administration episodes, depending on the results of the assessment. Because the actual act of causing the ingestion of the composition will in most cases be performed by the subject who is suffering from burns himself or herself, “administering” the composition to the subject is intended to embrace providing instructions to the subject to ingest the composition (such as might be done by a physician, physician's assistant, nurse, or any other health professional or consultant).

The present invention is further defined in the following Examples. It should be understood that these examples, while indicating preferred embodiments of the invention, are given by way of illustration only, and should not be construed as limiting the appended claims From the above discussion and these examples, one skilled in the art can ascertain the essential characteristics of this invention, and without departing from the spirit and scope thereof, can make various changes and modifications of the invention to adapt it to various usages and conditions.

### EXAMPLES

### Example 1

A clinical study was conducted in order to assess the effect of the administration of the inventive compositions to patients with type 2 diabetes and inadequate glycemic control (A1C>7.0%-<10.5%) despite oral medication therapy consisting of metformin >850 mg bid and another anti-hyperglycemic agent, versus placebo having similar carbohydrate, protein, fat and calories to that of the experimental composition.

Subjects were screened using a study design protocol approved by IRB. In each of three separate study centers, 24 patients were recruited for a double-blind, randomized, parallel-group study. Week 12 was selected as the primary time point. Patients meeting eligibility criteria were randomized to receive an embodiment of the inventive compositions or placebo 2×25 grams per day, half an hour before breakfast and half an hour before dinner, adjunct to their diabetic medicines. The compositions were administered in the form of a powdered beverage mixture combined with water. The analysis of covariance (ANCOVA) model was used for assessing statistically significant difference between the inventive composition and placebo on the change from baseline values of A1C, fasting blood glucose levels, body weight, BMI, lipid panel, blood pressure, and kidney function tests. Baseline value and age as covariates were used for assessing statistically significant difference between the inventive composition and the placebo.

Enrolled patients were aged 28 to 78 years undergoing therapy consisting of a single anti-hyperglycemic agent (either metformin or glibenclamide) or dual oral combination treatment (metformin and glibenclamide). Primary end point included A1C change from baseline after 12 weeks. Secondary end point included fasting blood glucose change from baseline after 12 weeks. Additionally, the study involved assessment of safety variables (change from baseline after 12 weeks) that included weight, BMI, total cholesterols, HDL, LDL, triglycerides, urea, uric acid, micro albumin, and creatinine. Finally, study included reporting of any adverse events during the experimental period.

Results—Mean Changes in Primary, Secondary End Points and Safety Variables at Week 12 from the Baseline in Patients with Type 2 Diabetes and Undergoing Oral Therapy with Metformin and Glibenclamide.

The analysis of covariance (ANCOVA) model with baseline value and age as covariates was used for assessing statistically significant difference between the inventive composition and placebo on the change from baseline values of A1c, fasting blood glucose levels, and aforementioned safety variables. Table 1, below, provides baseline characteristics for all patients enrolled in the study.

TABLE 1

| | | Experi- | | 95% Confidence Interval | | |
|---|---|---|---|---|---|---|
| Measures | Placebo | mental | Difference | Lower | Upper | p-Value |
| HbA1c | 8.45 | 9.26 | 0.81 | −193 | 1.817 | 0.1112 |
| Fasting Blood | 171.82 | 182.24 | 10.43 | −20.461 | 41.326 | 0.5006 |
| Glucose | | | | | | |
| Body | 68.67 | 71.64 | 2.97 | −3.697 | 9.64 | 0.376 |
| Weight(kg) | | | | | | |
| Body Mass | 29.22 | 30.16 | 0.94 | −1.942 | 3.827 | 0.5157 |
| Index | | | | | | |
| Total | 187.42 | 196.30 | 8.88 | −10.572 | 28.332 | 0.3644 |
| Cholesterol | | | | | | |
| HDL | 33.84 | 40.42 | 6.59 | −0.621 | 13.795 | 0.0723 |
| LDL | 106.57 | 119.57 | 13.18 | −8.944 | 35.301 | 0.2362 |
| Triglycerides | 196.332 | 236.84 | 40.51 | −34.154 | 115.175 | 0.2815 |
| SBP | 119.33 | 124.14 | 4.8 | −2.469 | 12.078 | 0.1912 |
| DBP | 75.33 | 77.93 | 2.6 | −2.098 | 7.294 | 0.2726 |
| Urea | 25.50 | 28.28 | 2.78 | −2.117 | 7.676 | 0.2603 |
| Uric Acid | 4.55 | 4.11 | −0.44 | −1.279 | 0.407 | 0.3047 |
| Micro | 28.21 | 33.45 | 5.23 | −17.729 | 28.197 | 0.6496 |
| albumin | | | | | | |
| Creatinine | 0.75 | 0.84 | 0.09 | −0.308 | 0.497 | 0.6378 |

Table 2, below, provides the results of the assessment of safety variables in subjects who were administered the experimental composition, as compared with the subjects who received a placebo composition.

TABLE 2

| | Experimental | | Placebo | | Experimental vs. Placebo | | |
|---|---|---|---|---|---|---|---|
| | (n = 18) | | (n = 11) | | Difference | | |
| Safety | | 12 | | 12 | (95% CI) | | |
| Variable | Baseline | Weeks | Baseline | Weeks | Lower to Upper | | p-Value |
| Weight (Kg) | 69.972 | 69.222 | 64.891 | 65.273 | 1.13 | (−1.564 to 3.828) | 0.4034 |
| BMI | 28.843 | 28.58 | 25.959 | 26.055 | 0.36 | (−0.831 to 1.549) | 0.5482 |
| Total | 190.564 | 184.564 | 200.465 | 194.306 | 0.38 | (−26.777 to 27.533) | 0.9778 |
| Cholesterol | | | | | | | |
| HDL | 36.28 | 51.78 | 38.129 | 42.507 | 11.61 | (−10.923 to 34.140) | 0.3048 |
| LDL | 112.736 | 118.326 | 120.366 | 123.666 | 1.80 | (−28.569 to 32.162) | 0.9054 |
| Triglycerides | 181.25 | 154.35 | 236.335 | 193.459 | 16.11 | (−77.028 to 109.253) | 0.7298 |
| SBP | 118.182 | 117.273 | 125.556 | 118.333 | 6.30 | (−5.143 to 17.746) | 0.2749 |
| DBP | 75.455 | 77.778 | 77.222 | 77.778 | 2.17 | (−5.547 to 9.880) | 0.5757 |
| Urea | 26.827 | 25.345 | 28.95 | 31.444 | −3.96 | (−11.707 to 3.786) | 0.3098 |
| Uric acid | 4.918 | 4.073 | 4.471 | 4.735 | −1.11 | (2.278 to 0.064) | 0.0634 |
| Micro albumin | 55.556 | 51.111 | 46.667 | 37.778 | 4.16 | (−30.755 to 39.075) | 0.812 |
| Creatinine | 0.713 | 0.688 | 0.8 | 0.73 | 0.05 | (−0.519 to 0.609) | 0.8716 |

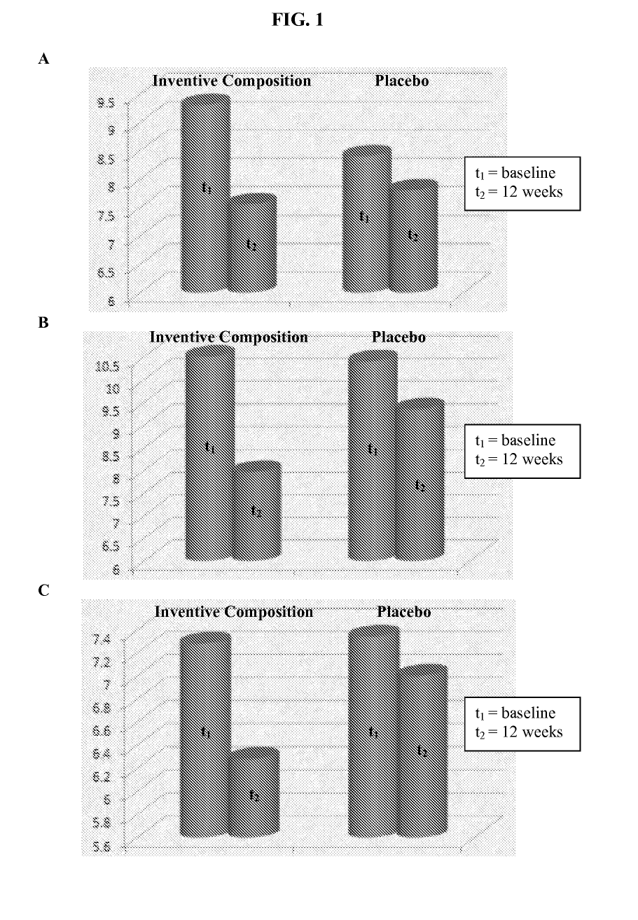

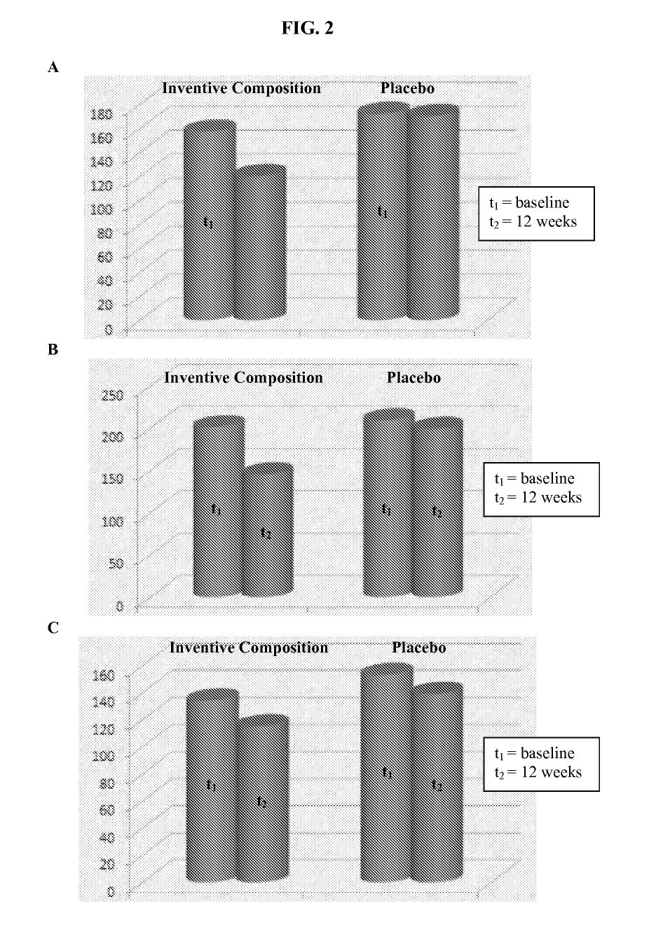

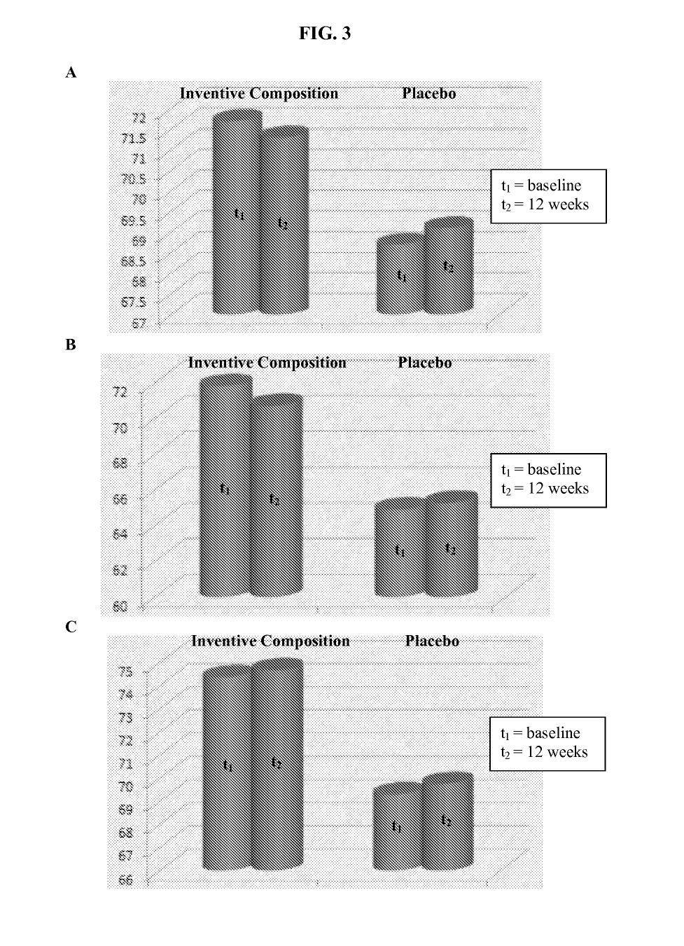

Tables 3 to 5, below, respectively show the mean change for primary end point (A1c) for all the patients and for the patients stratified at baseline to A1c>8.5. These results are also illustrated in FIGS. 1-3. FIG. 1 provides the results of a comparison between the effect of the experimental embodiment of the inventive compositions and the effect of the placebo composition on A1C test values in (A) all test subjects, (B) test subjects stratified to >8.5 A1C, and (C) test subject stratified to <8.5 A1C. FIG. 2 provides the results of a comparison between the effect of the experimental embodiment of the inventive compositions and the effect of the placebo composition on fasting blood glucose levels in (A) all test subjects, (B) test subjects stratified to >8.5 A1C, and (C) test subject stratified to <8.5 A1C. FIG. 3 provides the results of a comparison between the effect of the experimental embodiment of the inventive compositions and the effect of the placebo composition on patient weight in (A) all test subjects, (B) test subjects stratified to >8.5 A1C, and (C) test subject stratified to <8.5 A1C.

TABLE 3
Primary and Secondary End Points For All Patients

| | Estimate of | Estimate of | Placebo vs Treated | | | 95% Confidence | |
|---|---|---|---|---|---|---|---|
| | Placebo Mean | Treatment | Differ- | | | Interval | |
| | (N-30) | Mean (N-29) | ence | SE | pValue | Lower | Upper |
| HbA1c | −0.733 | −1.621 | −0.89 | 0.37 | 0.02 | −1.632 | −0.14 |
| Fasting | −5.389 | −38.956 | −33.6 | 15.81 | 0.039 | −65.38 | −1.75 |
| Blood | | | | | | | |
| Sugar | | | | | | | |

TABLE 4
Primary End Point (A1c) in Patients with A1c Greater Than 8.5%

| | Intervention, | | | | | |
|---|---|---|---|---|---|---|
| | n = 18 | 12 | Placebo = 11 | 12 | Placebo vs. Experimental | |
| Measures | Baseline | Weeks | Baseline | Weeks | Difference (95% CI) | p-Value |
| HbA1c % | 10.472 | 7.983 | 10.373 | 9.364 | −1.48 (0.45-2.508) | 0.0056 |

TABLE 5
Secondary End Point (Blood Glucose) in Patients with A1c Greater Than 8.5%

| | Intervention, | | | | | |
|---|---|---|---|---|---|---|
| | n = 18 | 12 | Placebo = 11 | 12 | Placebo vs Experimental | |
| Measures | Baseline | Weeks | Baseline | Weeks | Difference (95% CI) | p-Value |
| FBS | 201.125 | 144.688 | 198.8 | 209.6 | −67.28 (8.933-125.631) | 0.0248 |

Statistically significant reduction in A1c (−1.62%) was observed in all the patients to whom the inventive composition was administered compared to placebo in 12 weeks. There was a substantial drop in A1c, (−2.49%) in the patients in the experimental group whose A1c levels at baseline is greater than 8.5% compared to placebo. A similar trend for the secondary end point that is fasting blood glucose levels is observed, a decrease of mean fasting blood glucose levels (−38.9) for all the patients, 56.43 for the patients stratified at baseline to A1c>8.5.

No adverse events were reported and all patients exhibited good tolerance of administration of the inventive composition 2×25 grams per day.

The safety variables monitored during the study were within acceptable limits, and the general trend in patients to whom an inventive composition was administered was a modest weight loss, drop in cholesterol levels, significant decrease in systolic and diastolic blood pressure, and a marginal increase in triglycerides.

These results indicate that administration of a composition according to the present disclosure twice-daily was highly effective to provide statistically significant reduction in A1c and fasting blood glucose in patients with type 2 diabetes whose A1c was otherwise poorly controlled with oral medications. The inventive compositions provided substantial and additive glycemic improvement when added to metformin and sulfonyl urea treatment. The compositions appeared to be well tolerated in the subject population studied. Additional benefits included weight loss, and improvements in blood pressure, micro albumin and creatinine. The difference in lipid profiles for the inventive compositions was smaller as compared to the placebo.

### Example 2

### Protein, Peptide, and Amino Acid Content of Sample Embodiment

Proteins (wt %)

| | Embodiment 1 | Embodiment 2 |
|---|---|---|
| Whey Protein Isolate | 5.52 | 0 |
| Soy Protein Isolate | 2.7 | 0 |
| Soy Protein From Soy Milk | 19.76 | 20.14 |
| Powder | | |
| Instantized Calcium | 3.24 | 10.8 |
| Caseinate | | |
| Total | 31.22 | 30.94 |

Peptides (wt %)

| | Embodiment 1 | Embodiment 2 |
|---|---|---|
| Whey Protein Hydrolysate | 4.8 | 4.8 |
| Soy Protein Hydrolysate | 2.28 | 4.56 |
| Casein Hydrolysate | 2.55 | 0 |
| Total | 9.63 | 9.36 |

Amino Acids (wt %)

| | Embodiment 1 |
|---|---|
| Phenylalanine | 0.48 |
| Isoleucine | 0.48 |

### Example 3

### Sample Embodiment of the Inventive Compositions

| Ingredient | Range (wt %) |
|---|---|
| Soy Milk Powder | 40-60 |
| Alpha Lipoic Acid R + S | 0.33-3.3 |
| Alpha Lipoic Acid R-NA | 0.066-0.6 |
| Chromium Yeast | 0.4-3.2 |
| Dimagnesium Malate | 1.4-13.5 |
| Vitamin B6, Pyridoxin Hydrochloride | 0.02-0.18 |
| Vitamin E, DL-Alpha-Tocopheryl Acetate 50% | 0.27-2.4 |
| CWS Powder | |
| Vitamin C, Ascorbic Acid | 0.33-3.0 |
| Whey Protein Hydrolysate 8360 | 2.0-18.0 |
| Whey Protein Isolate 9010 | 2.0-18.1 |
| Soy Protein Hydrolysate | 1.0-9.0 |
| Soy Protein Isolate | 1.0-9.0 |
| Casein Hydrolysate | 1.0-9.0 |
| Instantized Calcium Caseinate | 1.2-10.8 |
| Cinnamon powder 10:1 | 0.27-2.4 |
| Cinnamon WS extracts | 0.08-0.75 |
| Resistant Starch (maltodextrin) | 2.05-24.6 |
| Aspartame | 0.57-5.16 |
| Biotin | 0.0067-0.06 |
| Cinnamon Flavor | 0.33-3.0 |
| Stabilizer | 0.216-1.95 |
| Isoleucine | 0.166-1.5 |
| Phenylalanine | 0.166-1.5 |
| Silicon dioxide | 0.166-1.5 |
| Potassium Citrate | 0.267-2.4 |
| Vitamin B12, Cyanocobalamin | 0.0000001-0.00001 |

### Example 4

### Sample Embodiment of the Inventive Compositions

| | | |
|---|---|---|
| Soy Milk Powder | 52 | Grams |
| Alpha Lipoic Acid R + S | 1 | Grams |
| Alpha Lipoic Acid R-NA | 0.2 | Grams |
| Chromium Yeast | 1 | Grams |
| Dimagnesium Malate | 4.4 | Grams |
| Vitamin B6, Pyridoxin Hydrochloride | 0.06 | Grams |
| Vitmain E, DL-Alpha-Tocopheryl Acetate 50% CWS | 0.8 | Grams |
| Powder | | |
| Vitamin C, Ascorbic Acid | 1 | Grams |
| Whey Protein Hydrolysate | 6 | Grams |
| Whey Protein Isolate | 6 | Grams |
| Soy Protein Hydrolysate | 3 | Grams |
| Soy Protein Isolate | 3 | Grams |
| Casein Hydrolysate | 3 | Grams |
| Instantized Calcium Caseinate | 3.6 | Grams |
| Cinnamon powder 10:1 | 0.8 | Grams |
| Cinnamon WS extracts | 0.25 | Grams |
| Resistant Starch (maltodextrin) | 8.2 | Grams |
| Aspartame | 1.72 | Grams |
| Biotin | 0.02 | Grams |
| Cinnamon flavor | 1 | Grams |
| Stabilizer | 0.65 | Grams |
| Isoleucine | 0.5 | Grams |
| Phenylalanine | 0.5 | Grams |
| Silicon dioxide | 0.5 | Grams |
| Potassium Citrate | 0.8 | Grams |
| Vitamin B12, Cyanocobalamin | 0.00001 | Grams |
| Total | 100 | Grams |

### Example 5

### Sample Embodiment of the Inventive Compositions

| | | |
|---|---|---|
| Soy Milk Powder | 53 | Grams |
| Alpha Lipoic Acid R + S | 1 | Grams |
| Alpha Lipoic Acid R-NA | 0.18 | Grams |
| Chromium Yeast | 1.2 | Grams |
| Dimagnesium Malate | 4.4 | Grams |
| Vitamin B6, Pyridoxin Hydrochloride | 0.06 | Grams |
| Vitamin E, DL-Alpha-Tocopheryl Acetate 50% CWS | 0.8 | Grams |
| Powder | | |
| Vitamin C, Ascorbic Acid | 1 | Grams |
| Whey Protein Hydrolysate | 6 | Grams |
| Soy Protein Hydrolysate | 6 | Grams |
| Instantized Calcium Caseinate | 12 | Grams |
| Cinnamon powder 4:1 | 1.275 | Grams |
| Cinnamon water-soluble extracts | 0.225 | Grams |
| Resistant Starch (maltodextrin) | 8 | Grams |
| Aspartame | 2 | Grams |
| Biotin | 0.09 | Grams |
| Cinnamon flavor | 1 | Grams |
| Stabilizer | 0.72 | Grams |
| Isoleucine | 0.5 | Grams |
| Phenylalanine | 0.5 | Grams |
| Total | 100 | Grams |

### Example 6

### Sample Embodiment of Liquid Beverage Comprising Inventive Composition

Ten to twelve ounces of water are poured in to a household blender or blender bottle. An aliquot (25 grams) of the composition as described in Examples 3, 4, or 5 are added, along with 2 tbsp of apple sauce, preferably without added sugar or of the sugar-free variety. Vanilla extract or any other desired flavor is added to taste. The combination is blended or shaken until smooth.

### Example 7

### Sample Embodiment of Liquid Beverage Comprising Inventive Composition

An aliquot (25 grams) of the composition as described in Examples 3, 4, or 5 is poured into a household blender or blender bottle, along with a serving of a desired fruit or fruit mixture, such as blueberries, blackberries, raspberries, strawberries, peaches, mangoes, or a combination thereof. Milk is added provide a desired consistency. The combination is blended or shaken until smooth.

### Example 8

### Sample Embodiment of Breakfast Food Comprising Inventive Composition

An ordinary serving size of oatmeal is prepared, for example, using ½ cup water or skim milk and 1 cup oatmeal. An aliquot (25 grams) of the composition as described in Examples 3, 4, or 5 are added, along with additional water or skim milk to provide a desired consistency.

### Example 9

### Sample Embodiment of Cookie Comprising Inventive Composition

Two tablespoons of water and two large eggs are beaten in mixing bowl until light and fluffy. An aliquot (25 grams) of the composition as described in Examples 3, 4, or 5 is added to the beaten mixture, along with one additional teaspoon cinnamon. Baking soda in the amount of ½ teaspoon, 1½ cups flour, and ¼ teaspoon salt are combined in a sifter, and about half of this combination is sifted over the egg mixture. The resulting mixture is folded to blend, and this process is repeated. The blended mixture is dropped in teaspoonful amounts onto greased cookie sheets, approximately 2 inches apart, and the dough is baked for 10 to 12 minutes at 375° F.

1. A composition for modulating the level of glycated hemoglobin or blood glucose in a subject comprising:
about 35 to about 65 wt % soy milk powder;
at least one additional protein;
at least two peptides each forming at least one of phenyalanine, leucine, isoleucine, valine, lysine, threonine, arginine, and 4-hydroxyisoleucine upon human metabolism;
at least one of phenyalanine, leucine, isoleucine, valine, lysine, threonine, arginine, and 4-hydroxyisoleucine;
at least one chromium-containing material;
at least one magnesium-containing material; and,
at least one antioxidant.

2. The composition according to claim 1 comprising whey protein or casein or a salt thereof.

3. The composition according to claim 1 comprising whey protein and casein or a salt thereof.

4. The composition according to claim 1 wherein said at least two peptides are respectively metabolic products of the soy protein in said soy milk powder and said at least one additional protein.

5. The composition according to claim 4 comprising any two of a), b) and c):
a) soy protein and soy protein hydrolysate;
b) casein or a salt thereof and casein hydrolysate;
c) whey protein and whey protein hydrolysate.

6. The composition according to claim 1 comprising one or more of phenylalanine, isoleucine, leucine, and valine.

7. The composition according to claim 1 comprising at least one fat-soluble antioxidant vitamin and at least one water-soluble antioxidant vitamin.

8. The composition according to claim 1 further comprising alpha lipoic acid.

9. The composition according to claim 8 comprising R alpha lipoic acid, R+S alpha lipoic acid, or both.

10. The composition according to claim 1 comprising vitamin D, vitamin E, or both.

11. The composition according to claim 1 comprising one or more of vitamin B6, vitamin B12, vitamin C, and biotin.

12. The composition according to claim 1 further comprising dietary fiber.

13. The composition according to claim 12 comprising a source of soluble fiber or a source of insoluble fiber.

14. The composition according to claim 1 further comprising a source of a polyphenol.

15. The composition according to claim 1 further comprising a viscosity modifier.

16. The composition according to claim 1 comprising:
about 35 to about 65 wt % soy milk powder;
about 0.35 to about 4.5 wt % alpha lipoic acid;
about 0.20 to about 5 wt % of a chromium-containing material;
about 1 to about 15 wt % of a magnesium-containing material;
about 0.40 to about 3.5 wt % of at least one water-soluble antioxidant vitamin;
about 0.20 to about 2.5 wt % of at least one water-insoluble antioxidant vitamin;
about 2 to about 18.5 wt % whey protein hydrolysate;
about 2 to about 18.5 wt % whey protein isolate;
about 1 to about 10 wt % soy protein hydrolysate;
about 1 to about 10 wt % soy protein isolate;
about 1 to about 10 wt % casein hydrolysate;
about 1 to about 11 wt % casein or a salt thereof;
about 2 to about 4 wt % of at least one of phenyalanine, leucine, isoleucine, valine, lysine, threonine, arginine, and 4-hydroxyisoleucine;
about 0.30 to about 3.5 wt % cinnamon powder or cinnamon extract; and,
about 2 to about 25 wt % dietary fiber.

17. The composition according to claim 1 comprising:
about 40 to about 60 wt % soy milk powder;
about 0.30 to about 3.5 wt % alpha lipoic acid R+S;
about 0.07 to about 0.60 wt % alpha lipoic acid R−Na;
about 0.40 to about 3.5 wt % of a chromium-containing material; about 1.2 to about 14 wt % of a magnesium-containing material; about 0.02 to about 0.20 wt % vitamin B6;
about 1.0E-8 to about 1.0E-5 vitamin B12
about 0.25 to about 2.5 wt % vitamin E;
about 0.30 to about 3 wt % vitamin C;
about 0.006 to about 0.06 wt % biotin;
about 2 to 18 wt % whey protein hydrolysate;
about 2 to about 18.5 wt % whey protein isolate;
about 1 to about 9 wt % soy protein hydrolysate;
about 1 to about 9 wt % soy protein isolate;
about 1 to about 9 wt % casein hydrolysate;
about 1 to about 11 wt % casein or a salt thereof;
about 0.25 to about 2.5 wt % cinnamon powder;
about 0.08 to about 0.75 wt % cinnamon extract;
about 2 to about 25 wt % of a source of soluble fiber;
about 0.15 to about 1.5 wt % isoleucine; and,
about 0.15 to about 1.5 wt % phenylalanine.

18. The composition according to claim 17 further comprising one or more of a sweetener, a flavoring agent, a buffering agent, a flow agent, and a stabilizer.

19. A powdered beverage mixture, liquid beverage, or solid food item comprising a composition according to claim 1.

20. A method for treating a glycated hemoglobin- or blood glucose-affected condition in a subject comprising administering to said subject a composition comprising
at least two proteins that undergo human metabolism at different rates, each forming one or more insulinotropic metabolic products upon human metabolism;
at least two peptides that undergo human metabolism at different rates, each forming one or more insulinotropic metabolic products upon human metabolism;
at least one insulinotropic amino acid, di-peptide, tri-peptide, or oligopeptide;
a biologically acceptable chromium-containing material;
a biologically acceptable magnesium-containing material; and, at least one antioxidant vitamin.

21. The method according to claim 20 wherein said condition is type 1 diabetes, type 2 diabetes, prediabetes, insulin resistance syndrome, metabolic syndrome, cystic fibrosis related diabetes, or polycystic ovarian syndrome.

22. The method according to claim 20 wherein said compound is administered to said subject as an adjunct therapy in combination with at least one other treatment for said condition.

23. The method according to claim 20 wherein said subject is undergoing insulin or oral medicine therapy for said condition at the time of the administration.

24. The method according to claim 20 wherein said composition further comprises alpha lipoic acid.

25. The method according to claim 20 wherein said composition further comprises dietary fiber.

26. A method for treating a subject that is suffering from burns, comprising administering to said subject a composition comprising
at least two proteins that undergo human metabolism at different rates, each forming one or more insulinotropic metabolic products upon human metabolism;
at least two peptides that undergo human metabolism at different rates, each forming one or more insulinotropic metabolic products upon human metabolism;
at least one insulinotropic amino acid, di-peptide, tri-peptide, or oligopeptide;
a biologically acceptable chromium-containing material;
a biologically acceptable magnesium-containing material; and,
at least one antioxidant vitamin.

27. A composition according to claim 1 in the form of a solid food item.
